(19) United States
(12) Patent Application Publication
(10) Pub. No.: US 2015/0305960 A1
(43) Pub. Date: Oct. 29, 2015

(54) MULTI-COMPARTMENTALIZED HAND REHABILITATION GLOVE

(71) Applicant: The Trustees of the Stevens Institute of Technology, Hoboken, NJ (US)

(72) Inventors: Tara Callahan, Harrisburg, PA (US); Lauren Hammerle, Sayville, NY (US); Ashley Princiotta, Manalapan, NJ (US); Becky Wos, Mukwonago, WI (US); Vikki Hazelwood, Wayne, NJ (US); Christian Gartner, Maywood, NJ (US)

(21) Appl. No.: 14/699,090

(22) Filed: Apr. 29, 2015

Related U.S. Application Data
(60) Provisional application No. 61/985,661, filed on Apr. 29, 2014.

(51) Int. Cl. A61H 1/00; A61F 7/00

(52) U.S. Cl. CPC A61H 1/008; A61F 7/00; A61F 2007/0036

(57) ABSTRACT

A system includes a thermal therapy element adapted to provide thermal therapy to a body part of a patient. The system also includes a compression therapy element adapted to provide compression therapy to the body part. The compression therapy element includes a plurality of portions. Each of the plurality of portions is adapted to be independently controllable to provide a corresponding one of a plurality of pressures to a corresponding one of a plurality of regions of the body part.

### CROSS-REFERENCE TO RELATED APPLICATION

The present application is a Section 111(a) application relating to and claiming the benefit of commonly owned, co-pending U.S. Provisional Patent Application No. 61/985,661, titled “MULTI-COMPARTMENTALIZED HAND REHABILITATION GLOVE,” having a filing date of Apr. 29, 2014, which is incorporated by reference herein in its entirety.

### FIELD OF THE INVENTION

The exemplary embodiments relate to rehabilitation devices and, more particularly, to hand rehabilitation compartments.

### BACKGROUND OF THE INVENTION

According to the CDC, over 3.7 million people visited the emergency room for a hand related injury in 2010. About 60% of these patients never regained normal hand function due to residual scar tissue. The average total cost of a hand injury was determined to be $6,162.76, including $246.96 from injury treatment and $5,915.80 from disability and lost wages. Rehabilitation for hand injuries may typically include controlled, directional compression that directs flow of endemic swelling out of the hand.

Current treatment techniques employ massage to direct the flow of edema toward the lymph for drainage. The dynamic compression sequence promotes the drainage of swelling toward the lymphatic drain in the patient's armpit. Reducing the amount of swelling decreases the inflammatory response that causes scar tissue formation, which may eventually lead to loss of hand function. Therefore, decreasing the inflammatory response reduces the amount of scar tissue formation, allowing patients to regain normal function in their hand.

However, existing wrap chambers consist of one compartment that progressively increases and releases pressure. Since these wraps consist of only one compartment, the current wraps cannot be tailored to the hand in a manner that effectively provides directional treatment and controlled physiological flow.

### SUMMARY OF THE INVENTION

In an embodiment, a system includes a thermal therapy element adapted to provide thermal therapy to a body part of a patient and a compression therapy element adapted to provide compression therapy to the body part. The compression therapy element includes a plurality of portions. Each of the plurality of portions is adapted to be independently controllable to provide a corresponding one of a plurality of pressures to a corresponding one of a plurality of regions of the body part. In an embodiment, the body part is a hand. In an embodiment, the body part is a foot. In an embodiment, the thermal therapy element includes a garment. In an embodiment, the garment includes a mitten. In an embodiment, the garment includes a glove.

In an embodiment, the thermal therapy element includes a first thermal therapy component adapted to placed on a first side of the body part and a second thermal therapy component adapted to be placed on a second side of the body part opposite the first side of the body part. In an embodiment, each of the first and second thermal therapy components includes a vessel containing a fluid. In an embodiment, the fluid is adapted to provide a therapeutically effective temperature.

In an embodiment, the compression therapy element comprises a first compression therapy component adapted to be placed on a first side of the body part and a second compression therapy component adapted to be placed on a second side of the body part opposite the first side of the body part. In an embodiment, the compression therapy element includes an air bladder. In an embodiment, the air bladder includes a plurality of compartments. In an embodiment, each of the plurality of compartments is adapted to correspond to one of the plurality of regions of the body part.

In an embodiment, the air bladder includes an air intake for receiving air from an external source. In an embodiment, the air intake includes a plurality of passages. In an embodiment, each of the plurality of compartments is adapted to receive air through a corresponding one of the plurality of passages. In an embodiment, the system also includes a pneumatic air pump coupled to the air intake. In an embodiment, the pneumatic air pump is configured to inflate and deflate the air bladder to produce a compression cycle for the body part.

In an embodiment, the compression cycle includes, in sequence, inflating a first one of the plurality of compartments, the first one of the plurality of components corresponding to a distal one of the regions of the body part; inflating a second one of the plurality of compartments, the second one of the plurality of components corresponding to a proximal one of the regions of the body part; deflating the second one of said plurality of compartments; and deflating the first one of the plurality of compartments.

In an embodiment, the compression cycle includes, in sequence, inflating a first one of the plurality of compartments, the first one of the plurality of components corresponding to a distal one of the regions of the body part; inflating a second one of the plurality of compartments, the second one of the plurality of components corresponding to a proximal one of the regions of the body part; deflating the first one of said plurality of compartments; and deflating the second one of the plurality of compartments.

In an embodiment, the compression cycle includes, in sequence, inflating a first one of the plurality of compartments, the first one of the plurality of components corresponding to a distal one of the regions of the body part; inflating a second one of the plurality of compartments, the second one of the plurality of components corresponding to a proximal one of the regions of the body part; deflating, substantially simultaneously, the first one and the second one of the plurality of components.

In an embodiment, the system also includes an enclosure housing the thermal therapy element and the compression therapy element.

### BRIEF DESCRIPTION OF FIGURES

FIG. 1 is a schematic representation of the elements of an exemplary hand rehabilitation device;

FIG. 2A is an illustration of a first exemplary thermal therapy element that may be employed by the hand rehabilitation device of FIG. 1;

FIG. 2B is an illustration of a second exemplary thermal therapy element that may be employed by the exemplary hand rehabilitation device of FIG. 1;

FIG. 2C is an illustration of a third exemplary thermal therapy element that may be employed by the exemplary hand rehabilitation device of FIG. 1;

FIG. 3A is a top perspective view of a first exemplary compression therapy element that may be employed by the exemplary hand rehabilitation device of FIG. 1;

FIG. 3B is a top perspective view of a second exemplary compression therapy element that may be employed by the exemplary hand rehabilitation device of FIG. 1;

FIG. 3C is an illustration of the compression therapy element of FIG. 3B as applied to the hand of a patient;

FIG. 4 is an illustration of an exemplary enclosure that may be employed by the exemplary hand rehabilitation device of FIG. 1;

FIG. 5 is an illustration of an exemplary hand rehabilitation device in preparation for use.

### DETAILED DESCRIPTION OF THE INVENTION

In an embodiment, the exemplary device may be used by the patient at home. In an embodiment, the exemplary device may be used in a clinical setting. In an embodiment, the device may be used to reduce swelling in the hands of patients with autoimmune disorders such as Rheumatoid arthritis. In an embodiment, the present invention may be adapted for use on other body parts than the hand. In an embodiment, the exemplary device may be adapted to fit the ankle or the foot. The exemplary embodiments will be described hereinafter with specific reference to the patient's hand, but it will be apparent to those of skill in the art that the same disclosure may be equally applicable to the patient's foot or to other body parts.

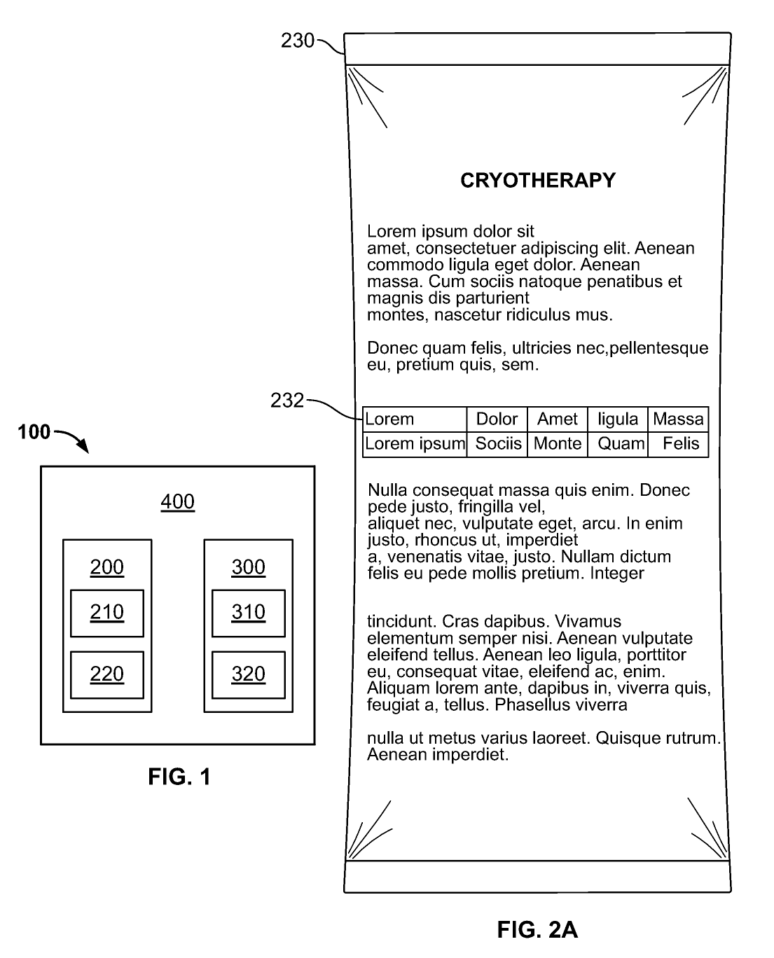

FIG. 1 schematically illustrates a device 100 according to the exemplary embodiments. In an embodiment, the device 100 includes three main functional elements, each of which may be implemented in various forms without departing from the broader principles of the exemplary embodiments. The first functional element of the device 100 is a thermal therapy element 200 providing a therapeutic temperature for thermal therapy (e.g., heat therapy or cold therapy) to the patient's hand during treatment. The second functional element of the device 100 is a compression therapy element 300 applying pressure to the patient's hand during treatment. The third functional element of the device 100 is an enclosure element 400 enclosing the patient's hand and part or all of the thermal therapy element 200 and the compression therapy element 300 during treatment. Each of the functional elements of the device 100 will be described in detail hereinafter. In some exemplary embodiments, two or three of the functional elements of the device 100 may be integrated into a unitary physical apparatus. In one exemplary embodiment, part of the compression therapy element 300 may be placed within the enclosure element 400 when the device 100 is in use and part of the compression therapy element 300 may remain outside the enclosure element 400.

The thermal therapy element 200 may include one or more separate physical components for providing a therapeutically effective temperature (e.g., heating or cooling) to the patient's hand. It will be known to those of skill in the art that either heat therapy, cold therapy, or both heat therapy and cold therapy at different times may be appropriate depending on the nature of the condition for which the patient is being treated. In an embodiment, the thermal therapy element 200 may include two components so that, during therapy, a first thermal therapy component 210 of the thermal therapy element 200 may be placed adjacent to a first side of the patient's hand (e.g., the palm of the patient's hand) and a second thermal therapy component 220 of the thermal therapy element 200 may be placed adjacent to a second side of the patient's hand (e.g., the back of the patient's hand). Each of the first and second components 210, 220 may be a vessel containing a gel or other fluid that is capable of providing a therapeutic temperature to the patient.

In an embodiment, each of the first and second components 210 and 220 of the thermal therapy element 200 may be a commercially manufactured gel pack 230, e.g., a pouch or other enclosure containing a gel or other flowable compound capable of being heated to hold a therapeutically effective warm temperature and/or cooled to hold a therapeutically effective cold temperature. The exemplary embodiments will be described herein with specific reference to a gel, but those of skill in the art will understand this disclosure to encompass other suitable substances capable of conforming to the patient's hand and of being used to perform thermal therapy as described herein. The exemplary embodiments of the thermal therapy element 200 may incorporate a gel that is capable of applying sufficient pressure in between the fingers and onto the intricate surface of the hand. Various gels and fluids may be incorporated into the exemplary embodiments, provided they are capable of withstanding and applying sufficient pressure.

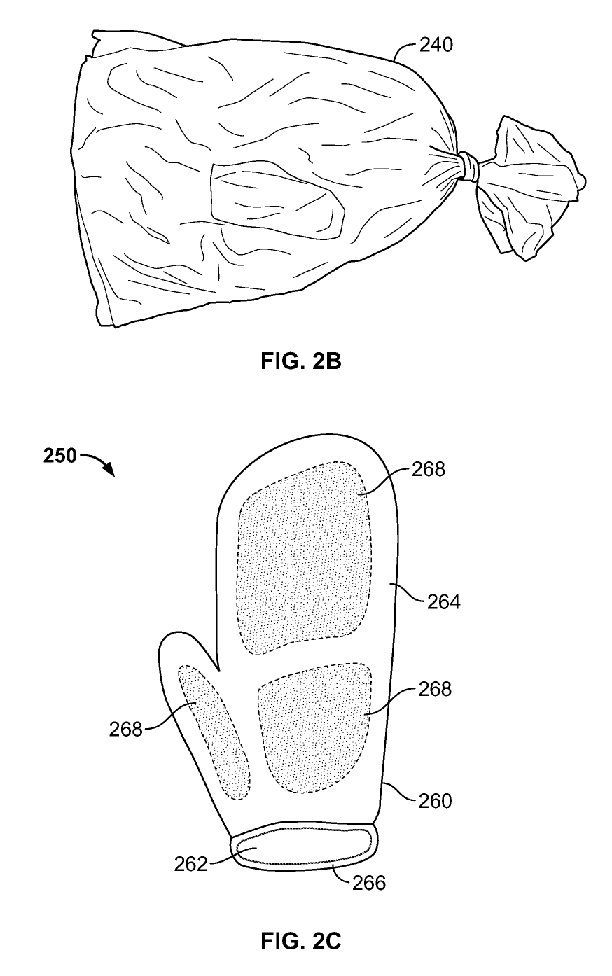

FIG. 2A illustrates an exemplary commercially manufactured gel pack 230 that may be used as one of the first and second components 210 and 220. The gel pack 230 may have instructions 232 printed thereon for instructing a user (e.g., the patient or a clinician) in the proper way to prepare the gel pack 230 for use in one or both of heat therapy and/or cold therapy. For example, the instructions 232 may instruct the user to place the gel pack 230 in a freezer for a specified period of time to prepare the gel pack 230 for use in cold therapy and/or place the gel pack 230 in a microwave oven for a specified period of time at specified power settings to prepare the gel pack 230 for use in heat therapy.

In another embodiment, with reference to FIG. 2B, each of the first and second components 210 and 220 of the thermal therapy element 200 may be a purpose-made gel pack 240 containing a gel or other flowable compound capable of heating or cooling as described above with the commercially manufactured gel pack 230. The gel pack 240 may be pliable in order to enable the gel contained therein to flow between the fingers of the patient, which may cause the therapy to be more effective. In an embodiment, the gel pack 240 may be less densely filled with gel than may be the case for the gel pack 230 to enable the gel pack 240 to better conform to the patient's hand. The gel pack 240 of FIG. 2B may be prepared for use in therapy in the same manner as described above with reference to the gel pack 230 of FIG. 2A, e.g., by storage in a freezer prior to cold therapy and/or heating in a microwave oven prior to heat therapy.

In another embodiment, the thermal therapy element 200 may include a single component adapted to provide thermal therapy to all targeted areas of a patient's hand. In an embodiment, the single component may include a pliable gel pack sized and shaped so as to cover both the upper and lower surfaces of the patient's hand. In an embodiment, with reference to FIG. 2C, the single component may include a garment 250 to be worn on the patient's hand and containing therein a gel or other appropriate substance for providing heat therapy or cold therapy. In an embodiment, the garment 250 may include a mitten 260 enclosing substantially all of the wearer's hand in a single compartment. In an embodiment, the garment 250 may include a glove having separate compartments for individual fingers of the wearer. In some cases, a mitten may be preferable to a glove in that a patient suffering from movement-impairing conditions such as arthritis may be unable to place his or her fingers in an appropriate position to wear a glove. In some cases, a glove may be preferable to a mitten in that a glove may enable thermal therapy to be delivered more closely to the areas between the patient's fingers. In an embodiment the garment 250 may be fabricated to fit hands of a desired size. In an embodiment, the garment 250 may be fabricated to fit a specific patient. In an embodiment, the garment 250 may be adjustable to fit a variety of patients.

FIG. 2C illustrates an exemplary garment 250 including a mitten 260. The mitten 260 may have a general shape in keeping with the common shape of a mitten. The mitten 260 may include an inner surface 262 that will be adjacent to the patient's hand when the mitten 260 is worn, and an outer surface 264 opposite the inner surface 262. The mitten 260 includes an opening 266 into which the patient's hand may be inserted. The mitten 260 also includes one or more compartments 268 disposed within the mitten 260, between inner surface 262 and outer surface 264, enclosing a gel appropriate for thermal therapy in a manner such that the gel is not exposed to the inner surface 262 or the outer surface 264, such that the gel does not come into direct contact with the skin of a patient who is wearing the mitten 260 or of a clinician who is administering therapy using the mitten 260, and such that the gel is retained within and does not escape from the compartments 268.

Those of skill in the art will be aware that the specific temperatures to be used for either heat therapy or cold therapy will vary depending on the nature of the patient's condition, the patient's tolerance for temperature, the location at which the temperature is to be measured, etc. Those of skill in the art will also be aware that the length of heat therapy or cold therapy to be performed using the thermal therapy element 200 may vary depending on the nature of the patient's condition, the patient's tolerance for temperature, etc. In an embodiment, either heat therapy or cold therapy may be performed for a time period of about ten minutes. Those of skill in the art will be aware that performance of heat therapy or cold therapy for longer time periods may cause damage to the patient's capillaries, reducing the effectiveness of the therapy and potentially causing harm to the patient. In an embodiment, a temperature of about 40 degrees Fahrenheit may be applied to the skin of the patient for cold therapy. In an embodiment, a temperature of about 98 to 113 degrees Fahrenheit may be applied to the skin of the patient for heat therapy. In an embodiment, a temperature of about 98 to 110 degrees Fahrenheit may be effective for use on a patient's hand or arm, and a temperature of about 98 to 104 degrees Fahrenheit may be effective for use on a patient's foot or leg. However, it will be apparent to those of skill in the art that the specific temperatures noted above are only exemplary, and that other temperatures may alternatively be chosen depending on factors such as the patient's tolerance for heat/cold, the therapist's judgment as to a therapeutically effective temperature, etc.

The compression therapy element 300 may include one or more separate physical components for providing a therapeutically effective pressure to the patient's hand. It will be known to those of skill in the art that the magnitude and duration of pressure to be applied may vary depending on the condition for which the patient is being treated. In an embodiment, referring back to FIG. 1, the compression therapy element 300 may include two pressure application components so that, during therapy, a first pressure application component 310 of the compression therapy element 300 may be placed adjacent to a first side of the patient's hand (e.g., the palm of the patient's hand) and a second pressure application component 320 of the compression therapy element 300 may be placed adjacent to a second side of the patient's hand (e.g., the back of the patient's hand). In an embodiment, the compression therapy element 300 may be adapted to apply differing pressures to different regions of the patient's hand. For example, each of the first and second pressure application components 310 and 320, respectively, may include a plurality of regions enabling such variations in applied pressure. In an embodiment, the compression therapy element 300 may also include a pressure generation component 330 that may generate pressure that may be conveyed to the pressure application components 310 and 320 (e.g., by means of pneumatic tubes or other appropriate conveyance) for application to the patient.

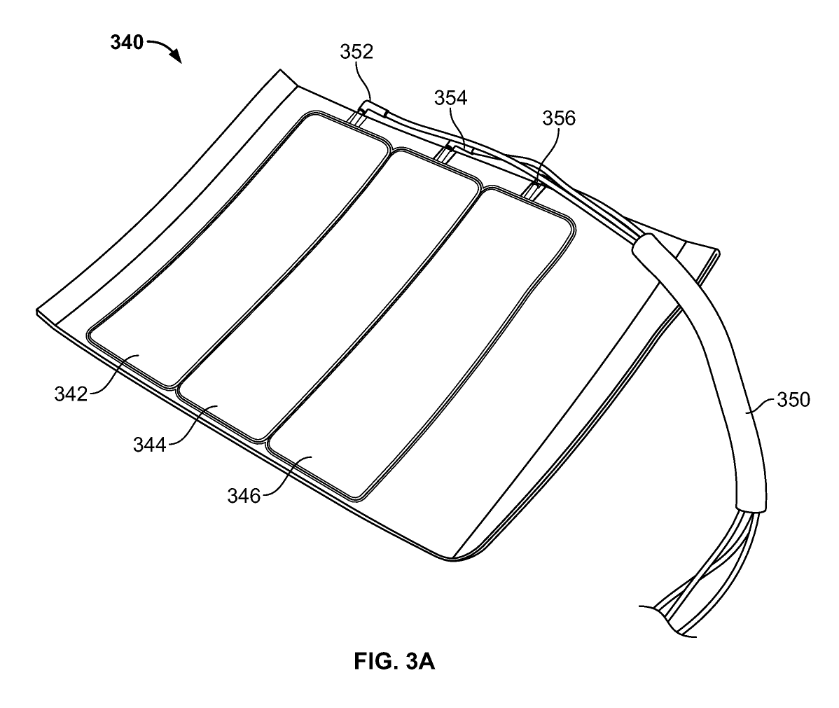

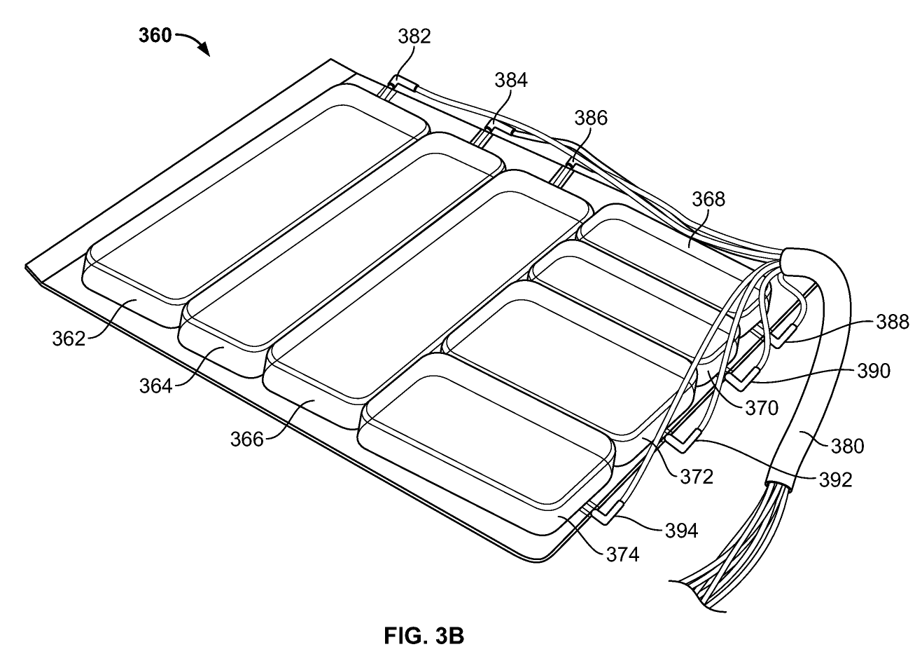

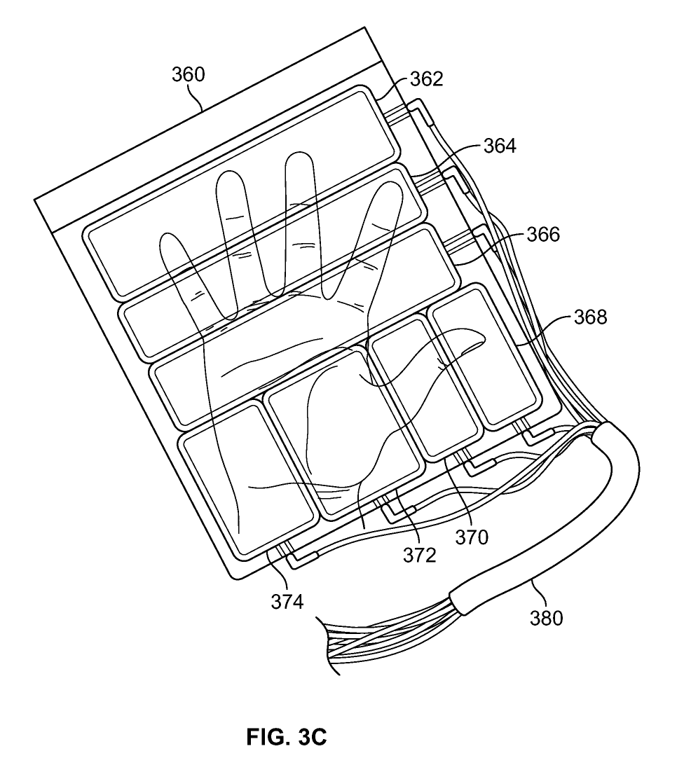

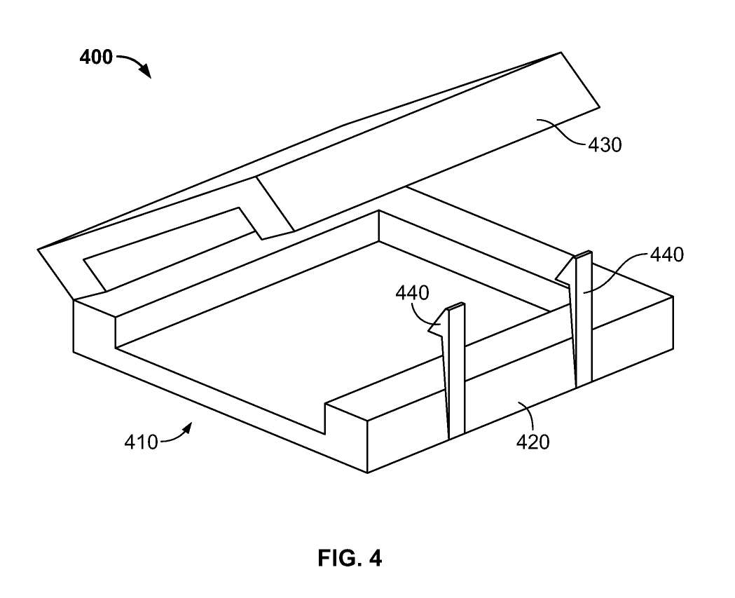

FIG. 3A illustrates an exemplary air bladder 340 that may function as one of the pressure application components 310 or 320. In an embodiment, one air bladder 340 may be used as the first pressure application component 310 and another air bladder 340 may be used as the second pressure application component 320. In an embodiment, the air bladder 340 may be sized and shaped so as to cover the entirety of a patient's hand. In an embodiment, the air bladder 340 may be sized and shaped so as to cover the entirety of the patient's hand and additionally to cover a portion of the patient's wrist. In an embodiment, the air bladder 340 may be fabricated to fit hands of a desired size. In an embodiment, the air bladder 340 may be fabricated to fit a specific patient. In an embodiment, the air bladder 340 may be “one-size-fits-all” to fit a variety of patients.

The exemplary air bladder 340 includes three compartments 342, 344, 346, that may be inflated with air to provide pressure to the patient's hand when the air bladder 340 is placed in proximity to the patient's hand. The air bladder 340 may be fabricated from an appropriate material (e.g., rubber, plastic, etc.) such that significant quantities of air do not escape the air bladder 340 through the material. The air bladder 340 may be placed along the patient's hand such that each of the compartments 342, 344, 346 may overlap a different portion of the hand. For example, compartment 342 may be in proximity to the fingertips, compartment 344 may be in proximity to the lower portions of the fingers, the tip of the thumb, and the upper palm, and compartment 346 may be in proximity to the base of the thumb and the lower palm.

Each one of the three compartments 342, 344, 346 is capable of being filled with air to varying degrees in order that they may be capable of providing varying levels of pressure. For example, compartment 342 may be capable of being filled with a varying amount of air in order to provide varying pressure to, for example, the fingertips. Further, the compartments 342, 344, 346 may be capable of being filled with differing amounts of air than one another in order to provide different levels of pressure to different areas of the patient's hand. For example, it may be desirable to inflate compartment 342 with air to a greater pressure than compartment 346, and thereby apply greater pressure to the patient's fingertips than to the patient's palm. It will be apparent to those of skill in the art that this pressure variation is only exemplary and that the specific variation of pressure to be applied may vary for each individual patient and/or during the course of an individual patient's treatment. It will further be apparent to those of skill in the art that the inclusion of three compartments 342, 344, 346 is only exemplary, and that greater or smaller numbers of compartments may be included in different embodiments. In an embodiment, four compartments may be present. In an embodiment, five compartments may be present. In an embodiment, six compartments may be present. In an embodiment, seven compartments may be present. The inclusion of a greater number of compartments may be desirable because it may provide for a greater level of control of the flow of endemic fluid out of the hand, as will be described hereinafter.

The air bladder 340 may include an air intake 350, which may itself be made up of a plurality of passages 352, 354, 356. Each of the passages 352, 354, 356 may correspond to one of the compartments 342, 344, 346, and may supply air to its corresponding compartment in order to apply pressure as described herein. Thus, it will be apparent to those of skill in the art that the specific presence of three passages 352, 354, 356 in the present disclosure is only exemplary, and that other quantities of passages may be present in different embodiments. The air intake 350 may be adapted to be connected to, and receive a supply of air from, a pneumatic air pump in order to inflate compartments 342, 344, 346 of the air bladder 340 to a desired pressure. In an embodiment, air intake 350 may be adapted to be connected to an SC-3004-FC sequential circulator manufactured by Bio Compression Systems, Inc., of Moonachie, N.J. However, it will be apparent to those of skill in the art that this specific adaptation is only exemplary, and that air intake 350 may be adapted to receive an air input from any air pump capable with supplying the air bladder 340 with appropriate therapeutic pressure as described herein.

FIG. 3B illustrates a second exemplary air bladder 360 that may also, as an alternative to air bladder 340, function as one of the pressure application components 310 or 320. In an embodiment, one air bladder 360 may be used as the first pressure application component 310 and another air bladder 360 may be used as the second pressure application component 320. In an embodiment, the air bladder 360 may be sized and shaped so as to cover the entirety of a patient's hand. In an embodiment, the air bladder 360 may be sized and shaped so as to cover the entirety of the patient's hand and additionally to cover a portion of the patient's wrist. In an embodiment, the air bladder 360 may be fabricated to fit hands of a desired size. In an embodiment, the air bladder 360 may be fabricated to fit a specific patient. In an embodiment, the air bladder 360 may be “one-size-fits-all” to fit a variety of patients.

With reference to FIG. 3C, the exemplary air bladder 360 includes seven compartments 362, 364, 366, 368, 370, 372, 374 that may be inflated with air to provide pressure to the patient's hand when the air bladder 360 is placed in proximity to the patient's hand. The air bladder 360 may be fabricated from an appropriate material (e.g., rubber, plastic, etc.) such that significant quantities of air do not escape the air bladder 360 through the material. The air bladder 360 may be placed along the patient's hand such that each of the compartments 362, 364, 366, 368, 370, 372, 374 may overlap a different portion of the hand, as shown in FIG. 3C. For example, compartment 362 may overlap the fingertips, compartment 364 may overlap the lower fingers, compartment 366 may overlap portions of the palm proximate to the fingers, compartment 368 may overlap the tip of the thumb, compartment 370 may overlap the lower thumb, compartment 372 may overlap the scaphoid region of the palm closest to the thumb, and compartment 374 may overlap the region of the palm closest to the ulna. It will be apparent to those of skill in the art that it may be desirable to apply a lower level of pressure to the scaphoid region than to other parts of the palm due to the large number of nerves present in this region.

Each one of the seven compartments 362, 364, 366, 368, 370, 372, 374 is capable of being filled with air to varying degrees in order that they may be capable of providing varying levels of pressure. For example, compartment 362 may be capable of being filled with a varying amount of air in order to provide varying pressure to, for example, the fingertips. Further, the compartments 362, 364, 366, 368, 370, 372, 374 may be capable of being filled with differing amounts of air than one another in order to provide different levels of pressure to different areas of the patient's hand. For example, it may be desirable to inflate compartment 362 with air to a greater pressure than compartment 366, and thereby apply greater pressure to the patient's fingertips than to the patient's palm. It will be apparent to those of skill in the art that this pressure variation is only exemplary and that the specific variation of pressure to be applied may vary for each individual patient and/or during the course of an individual patient's treatment. As noted above with reference to the air bladder 340, it will further be apparent to those of skill in the art that the inclusion of seven compartments 362, 364, 366, 368, 370, 372, 374 of the air bladder 360 is only exemplary, and that greater or smaller numbers of compartments may be included in different embodiments.

The air bladder 360 may include an air intake 380, which may itself be made up of a plurality of passages 382, 384, 386, 388, 390, 392, 394. Each of the passages 382, 384, 386, 388, 390, 392, 394 may correspond to one of the compartments 362, 364, 366, 368, 370, 372, 374, and may supply air to its corresponding compartment in order to apply pressure as described herein. Thus, it will be apparent to those of skill in the art that the specific presence of seven passages 382, 384, 386, 388, 390, 392, 394 in the present disclosure is only exemplary, and that other quantities of passages may be present in different embodiments. The air intake 380 may be adapted to be connected to, and receive a supply of air from, a pneumatic air pump in order to inflate compartments 362, 364, 366, 368, 370, 372, 374 of the air bladder 360 to a desired pressure.

FIG. 4 illustrates an exemplary enclosure element 400 that may house the thermal therapy element 200 and the compression therapy element 300 during treatment. In the embodiment illustrated in FIG. 4, the enclosure element 400 includes a plastic housing 410 including a lower portion 420 and an upper portion 430 that are connected by one or more hinges (not shown); the housing 410 is closed by closure means 440. In the embodiment shown in FIG. 4, the closure means 440 include clasps; in other embodiments, the closure means 440 may include hook-and-loop fasteners or any other suitable closure means. In an embodiment, ⅛″ thick Lexan pieces may be laser cut and glued together using Lexan polycarbonate glue IPS 40 PT to form two rectangular boxes that are used in fabricating the enclosure element 400. It will be apparent to those of skill in the art that the exemplary enclosure element 400 shown in FIG. 4 is only one possible enclosure, and that variations may be possible without departing from the general principle of the enclosure element 400. In another embodiment, the overall design of the exemplary embodiments may be made more compact provided that sufficient pressure is capable of being applied to a patient's body. In another embodiment, the enclosure element 400 may have curved edges to ensure comfort for the patient. Other embodiments may incorporate a sequential circulator into the enclosure element 400 to make the device more portable for clinical and at-home use. In an embodiment, the area of casing around the finger tips may be decreased to allow for better distribution of pressure between the finger tips. In a further embodiment, the enclosure element 400 may be fabricated by 3-D printing or by using a different material. In an embodiment, enclosure element 400 may be manufactured by beginning with a thicker sheet and hollowing out the area for the rectangular concave mold.

The following steps outline how the exemplary embodiments may be used to dynamically drain swelling from a patient's hand. The exemplary technique will be described herein with specific reference to the elements of the exemplary device 100, but those of skill in the art will understand that this technique may also be adapted to application with varying structural elements. Initially, the patient's hand is placed into the device 100. The specific steps for this insertion may vary based on the specific structural elements of the device 100. For example, in an embodiment where thermal therapy element 200 includes a garment, such as mitten 260, the garment may be placed onto the patient's hand. The hand, wearing the garment such as mitten 260, may then be inserted into the enclosure element 400. Alternatively, in an embodiment where thermal therapy element 200 includes separate components 210, 220, a first component 210 (e.g., gel bag 240) may be placed below the patient's hand, and a second component 220 (e.g., gel bag 240) may be placed above the patient's hand. Gel contained within first component 210 and second component 220 may surround the patient's hand and flow between the fingers. In either case, prior to application to the patient's hand, the thermal therapy element 200 may be heated or cooled to a desired therapeutic temperature depending on the treatment protocol for that patient, such as by placement in a freezer or heating in a microwave oven.

Compression therapy element 300 may then be applied to the patient's hand. In an embodiment, a first pressure application component 310 may be placed below the patient's hand, and a second pressure application component 320 may be placed above the patient's hand. Each of the pressure application components 310, 320 may be, for example, air bladder 360 described above. A supply of air may be connected to the pressure application components 310, 320, such as by connecting a pneumatic pump to air intakes 380 of air bladders 360 serving as pressure application components 310, 320. The enclosure element 400 may then be closed, with the patient's hand, the thermal therapy element 200, and the compression therapy element 300 enclosed therein. In an embodiment, the enclosure element 400 may act to retain the compression therapy element 300 in a suitable position to apply pressure to the patient's hand. Enclosure element 400 may be secured in a closed position, such as by fastening closure means 440 of housing 410.

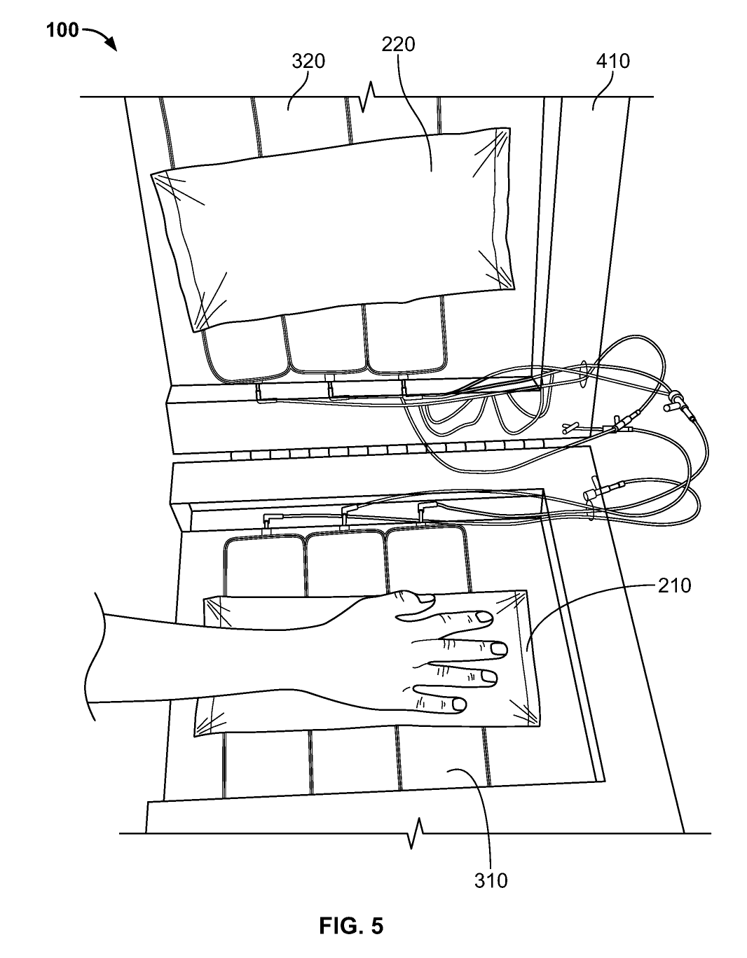

FIG. 5 illustrates the application of the device 100 to a patient. The lower portion 420 of enclosure 410 supports first pressure application component 310 (e.g., air bladder 340) below first thermal therapy component 210 (e.g., gel pack 230). The patient's hand may rest on the first thermal therapy component 210. The upper portion 430 of enclosure 410 encloses second pressure application component 320 (e.g., a second air bladder 340) and second thermal therapy component 220 (e.g., a second gel pack 230) in an arrangement such that, when the enclosure 410 is closed, second thermal therapy component 220 will be adjacent to the upper side of the patient's hand.

Once the patient's hand is properly inserted into the device 100, compression therapy may begin. It will be known to those of skill in the art that the precise nature of the compression therapy may vary depending on factors including the patient's condition, the clinician's planned course of treatment, the patient's stage of progress through a planned course of treatment, etc. Compression therapy will be described with reference to exemplary air bladder 340, but those of skill in the art will understand that the principles embodied by the following description may also be adapted to the air bladder 360 or to a different version of the compression therapy element 300.

Compression therapy may involve sequential inflation and deflation of the compartments 342, 344, 346 of the air bladder 340. This sequential inflation and deflation may be accomplished through configuration of a pneumatic air pump connected to air bladder 340 via air intake 350. In a first exemplary sequence, compartment 342, closest to the patient's fingertips, may be inflated first. Next, compartment 344, closest to the base of the patient's fingers and the upper palm, may be inflated. Next, compartment 346, closest to the base of the patient's palm, may be inflated. Next, compartment 342 may be deflated. Next, compartment 344 may be deflated. Next, compartment 346 may be deflated.

In a second exemplary sequence, compartment 342, closest to the patient's fingertips, may be inflated first. Next, compartment 344, closest to the base of the patient's fingers and the upper palm, may be inflated. Next, compartment 346, closest to the base of the patient's palm, may be inflated. Next, compartments 342, 344, 346 may be deflated simultaneously.

In a third exemplary sequence, compartment 342, closest to the patient's fingertips, may be inflated first. Next, compartment 344, closest to the base of the patient's fingers and the upper palm, may be inflated. Next, compartment 346, closest to the base of the patient's palm, may be inflated. Next, compartment 346 may be deflated. Next, compartment 344 may be deflated. Next, compartment 342 may be deflated.

In each case, the compartments 342, 344, 346, of each air bladder 340 (e.g., a first air bladder 340 serving as first pressure application component 310 and a second air bladder 340 serving as second pressure application component 320) may inflate and deflate according to the same sequence. In each sequence, each of the compartments 342, 344, 346 may be inflated to a pressure selected by a clinician and suitable for application to the corresponding region of the hand. Pressures may be selected so as not to prevent the patient from losing blood circulation within the hand. All pressures may be less than the patient's diastolic blood pressure in order to prevent this from occurring. Pressures may be selected to be greater near an injury and/or the patient's fingertips and less near the patient's wrist. Portions of the patient's hand closer to the fingertips may be referred to herein as distal portions of the hand; portions of the patient's hand closer to the wrist may be referred to herein as proximal portions of the hand.

In some cases, a treatment session may include a plurality of cycles of a compression sequence as described above. For example, a cycle of the first, second, or third compression sequence may take place over a time interval (e.g., one minute, five minutes, etc.), and the sequence may be repeated for a plurality of cycles (e.g., ten cycles, fifteen cycles, twenty cycles, etc.) over the course of a treatment session. A treatment session may include relaxation periods with no applied pressure during each pressure cycle. In an embodiment, each cycle may last one minute, and the cycles may be repeated fifteen to twenty times. Alternatively, the air bladder 340 may be inflated at the start of a treatment session and remain inflated for the entire length of a treatment session (e.g., an hour).

As described above, thermal therapy may be performed for a time period of, for example, ten minutes. However, a compression therapy session may be performed for a time period of, for example, an hour. In such a case, the compression therapy session may be briefly interrupted to allow for completion of the thermal therapy session. For example, with reference to the system shown in FIG. 5, enclosure 410 may be opened, first and second thermal therapy components 210 and 220 may be removed, enclosure 410 may be closed, and compression therapy may continue. Alternately, the thermal therapy element 200 (e.g., first and second thermal therapy components 210 and 220) may simply be allowed to remain in contact with the patient, and will naturally come to equilibrium at a temperature roughly equivalent to the patient's body temperature.

Pressures used may vary during the course of a treatment session; for example, pressure may be set at a low level at the start of a treatment session and increase as treatment progresses. Pressures used may also vary during a course of treatment including multiple treatment sessions; for example, pressures may be set at a low level during a first session and increase during subsequent sessions as the patient becomes accustomed to the application of pressure.

While the steps of use have been described with reference to a particular exemplary embodiment of the present invention, it will be apparent to one of ordinary skill in the art that variations of the above embodiment are to be included within the scope of the invention. For instance, the present invention may include any number of gel packs, any number of air bladders and any number of compartmentalized regions contained within the air bladders. Further, inflation may be sequential or simultaneous and deflation may similarly be sequential or simultaneous; sequences for sequential inflation or deflation may vary from those described above. Further, the exemplary embodiments may be adapted to provide other types of therapy (e.g., electrical stimulation) while thermal and compression therapy are being provided. Finally, the exemplary embodiments are capable of adaptation for treatment of other body parts of the human body, such as, for instance, the foot, knee, elbow, arm, leg, etc., and any other area of the human body which would benefit from full surface area coverage, a reduction of swelling, and application of pressure in discrete regions.

The multiple compartments of the device of the exemplary embodiments allow for controlled, directional flow of the endemic fluid out of the patient's hand. The multiple compartments may also be synchronized to provide the physiological flow control needed to effectively remove the swelling from the patient's hand. Therefore, the exemplary embodiments are more medically efficacious in treating hand injuries and removing swelling from the patient's hand.

The exemplary embodiments are intended to be used to reduce swelling in a patient's hand by providing compression and thermal therapy that treat the entire surface of the hand with a multi-compartmentalized design that directs and controls the physiological flow of edema out of the hand. The reduction in swelling will reduce the amount of scar tissue formation in the hand and help a patient regain function, reducing the total cost of the injury and improving clinical outcomes.

The exemplary embodiments provide many benefits to the patient. The use of a thermal therapy element inside of a compression element may offer treatment to the entire hand and the affected area in all three dimensions. During compression sequences, gel contained within a thermal therapy element will have the ability to mold to the patient's hand. The gel will be able to provide thermal therapy to the crevices and isolated areas between the fingers. This will ensure the patient is receiving treatment to the injured area regardless of the location. The exemplary embodiments also incorporate dynamic treatment from the fingertips to the forearm, which enables swelling to be drained out of the hand. Since a patient is often unaware of his or her pain threshold and may become uncomfortable during treatment, the pressure may be immediately modified during the treatment cycle. Additionally, in embodiments using a clear plastic housing, a clinician may be able to view the patient's hand throughout the treatment, preventing further damage that may be caused by poor circulation and improving the patient's safety. The exemplary embodiments may promote faster, more effective recovery, reduce the amount of scar tissue formation in the hand, and help a patient regain function, improving clinical outcomes. The exemplary embodiments may also reduce the total cost of an injury by helping the patient return to work more quickly, reducing health care costs, and decreasing the amount of money lost from disability or unemployment.

It should be understood that the embodiments described herein are merely exemplary in nature and that a person skilled in the art may make many variations and modifications thereto without departing from the scope of the present invention. All such variations and modifications, including those discussed above, are intended to be included within the scope of the invention.

What is claimed is:

1. A system, comprising:
a thermal therapy element adapted to provide thermal therapy to a body part of a patient; and
a compression therapy element adapted to provide compression therapy to the body part, said compression therapy element including a plurality of portions, each of said plurality of portions being adapted to be independently controllable to provide a corresponding one of a plurality of pressures to a corresponding one of a plurality of regions of the body part.

2. The system of claim 1, wherein the body part is a hand.

3. The system of claim 1, wherein the body part is a foot.

4. The system of claim 1, wherein said thermal therapy element comprises a garment.

5. The system of claim 4, wherein said garment comprises a mitten.

6. The system of claim 4, wherein said garment comprises a glove.

7. The system of claim 1, wherein said thermal therapy element comprises a first thermal therapy component adapted to placed on a first side of the body part and a second thermal therapy component adapted to be placed on a second side of the body part opposite the first side of the body part.

8. The system of claim 7, wherein each of said first and second thermal therapy components comprises a vessel containing a fluid.

9. The system of claim 8, wherein said fluid is adapted to provide a therapeutically effective temperature.

10. The system of claim 1, wherein said compression therapy element comprises a first compression therapy component adapted to be placed on a first side of the body part and a second compression therapy component adapted to be placed on a second side of the body part opposite the first side of the body part.

11. The system of claim 1, wherein said compression therapy element comprises an air bladder.

12. The system of claim 11, wherein said air bladder comprises a plurality of compartments, each of said plurality of compartments being adapted to correspond to one of the plurality of regions of the body part.

13. The system of claim 12, wherein said air bladder includes an air intake for receiving air from an external source.

14. The system of claim 13, wherein said air intake comprises a plurality of passages, and wherein each of said plurality of compartments is adapted to receive air through a corresponding one of said plurality of passages.

15. The system of claim 13, further comprising a pneumatic air pump coupled to said air intake.

16. The system of claim 15, wherein said pneumatic air pump is configured to inflate and deflate said air bladder to produce a compression cycle for the body part.

17. The system of claim 16, wherein said compression cycle comprises, in sequence:
inflating a first one of said plurality of compartments, said first one of said plurality of components corresponding to a distal one of the regions of the body part;
inflating a second one of said plurality of compartments, said second one of said plurality of components corresponding to a proximal one of the regions of the body part;
deflating said second one of said plurality of compartments; and
deflating said first one of said plurality of compartments.

18. The system of claim 16, wherein said compression cycle comprises, in sequence:
inflating a first one of said plurality of compartments, said first one of said plurality of components corresponding to a distal one of the regions of the body part;
inflating a second one of said plurality of compartments, said second one of said plurality of components corresponding to a proximal one of the regions of the body part;
deflating said first one of said plurality of compartments; and
deflating said second one of said plurality of compartments.

19. The system of claim 16, wherein said compression cycle comprises, in sequence:
inflating a first one of said plurality of compartments, said first one of said plurality of components corresponding to a distal one of the regions of the body part;
inflating a second one of said plurality of compartments, said second one of said plurality of components corresponding to a proximal one of the regions of the body part; and
deflating, substantially simultaneously, said first one and said second one of said plurality of compartments.

20. The system of claim 1, further comprising an enclosure housing said thermal therapy element and said compression therapy element.
